(19) United States
(12) Patent Application Publication
(10) Pub. No.: US 2024/0331842 A1
(43) Pub. Date: Oct. 3, 2024

(54) Digital Technology Enhancing Health and Well-Being Through Collective Mindfulness Practices Powered by Big Data

(71) Applicants: Alexey Ghidirim, Dubai (AE); Olga Alexandrovna Gidirim, Dubai (AE)

(72) Inventors: Alexey Ghidirim, Dubai (AE); Olga Alexandrovna Gidirim, Dubai (AE)

(21) Appl. No.: 18/619,994

(22) Filed: Mar. 28, 2024

Related U.S. Application Data
(60) Provisional application No. 63/455,012, filed on Mar. 28, 2023.

(51) Int. Cl. G16H 20/70; G16H 40/67

(52) U.S. Cl. CPC G16H 20/70; G16H 40/67

(57) ABSTRACT

The presented invention relates to educational and digital health technologies. A computer-implemented method of conducting group online practices, containing stages: the user's voice is recorded using the user's device; sending the recorded voice to the server for processing or performing processing locally on the user's device in order to assign the user to at least one group for performing practices; create at least one practice for a group of users based on the data received from the user; carry out the execution of the created practices in the group, and the execution of the practices is monitored by at least one user device or at least one wearable device of the user from the group and data is collected when performing the practices. The solution is aimed at creating a tool and conditions for conducting effective online group practices, where users have a feeling of the physical presence of the group.

### FIELD OF THE INVENTION

The presented invention relates to new educational and digital health technologies. This includes digital care programs, health, life and community technologies to improve health care delivery and well-being. It uses information and communications technology to make it easier to understand the health problems and challenges faced by people receiving social prescribing in a more personalized and accurate way.

The present invention represents a significant advance in the field of digital health and wellness. This innovative platform, combining advanced information and communication technologies, offers a comprehensive and integrated approach to healthcare delivery.

The system uses advanced algorithms and machine learning models to analyze various sources of information, including, but not limited to, voice, video and sensor data collected from mobile devices. This results in a highly personalized and accurate assessment of the health problems and challenges people face.

Technology aims to facilitate understanding of these issues through the application of digital care programs and the integration of multiple factors related to health, well-being, lifestyle and social influences.

The platform is a new approach to social prescribing, harnessing the power of technology to deliver personalized and effective solutions to improve health outcomes and overall well-being.

### INTRODUCTION

The field of educational and digital health technologies is relevant and interesting because it is shaping the future of medicine, making psychological and physiological care more accessible and widespread, and becoming more accurate and smart thanks to artificial intelligence algorithms. Intelligent programs and applications in the healthcare sector are of particular importance, since they can give every smartphone user the opportunity to have a smart assistant at hand, ready to provide high-quality and fast medical advice and support.

Recently, mobile meditation apps have begun to emerge, giving users the opportunity to listen to meditation, white noise, or nature sounds. The library consisted of relaxation practices that helped to relax and restore the user's psychological health.

However, meditations, podcasts and practices in such applications are not individually adapted to the user's personality, taking into account the emotional state. Users have to independently create a list of their favorite meditations.

Some of the new meditation apps have begun to create personal collections based on individual preferences based on selected meditations and practices, using neural network algorithms.

However, the problem with such applications is that the user can only interact with the program. There is no possibility of human communication, it is impossible to practice joint meditation. At the same time, social connections allow you to quickly restore psychological balance, and joint practices increase efficiency. Additionally, current apps have limited functionality and only focus on meditation.

Because of these shortcomings, there is a need for a product that would be not just a smart health application, but a virtual world for joint meditations of various users. It is a true mindfulness community where users can experience and practice a variety of techniques and practices alone or in groups, including audio practices, video practices, and walking practices. The present invention is a unique healthcare ecosystem aimed at creating a collective meditative experience that will help users maintain psychological and physiological health.

### BRIEF DESCRIPTION OF THE INVENTION

The present invention includes new digital technology and a set of methods designed to help people maintain healthy physical and emotional states. Users of this invention can easily track and improve their well-being through personal data collection, analysis and personalized recommendations.

The solution is aimed at creating a tool and conditions for conducting effective online group practices, where users have a feeling of the physical presence of the group.

The present invention can be run on iOS and Android platforms as well as on the Internet, and has its own database and API capabilities. It allows users to connect external devices for improved tracking and analysis of their personal health and wellness data.

Using advanced algorithms and personalized analytics, the invention provides users with personalized recommendations and advice on how to improve their overall health and well-being. By comparing a user's data with that of others, the invention can identify effective methods tailored to each individual's unique needs.

Apart from providing valuable information and advice, the invention can also prevent potential health problems by identifying potential risks and providing timely recommendations on how to eliminate them.

The proposed solution does:

1. Create and analyze personalized singing practices using a customizable feature. This feature allows users to choose from a variety of syllables including Om, Ill-lah, Sat-nam, On-nam, etc., or create their own combination of syllables.

2. Creates and analyzes expressive speech communication practices of gibberish monitored using a smartphone or wearable device.

3. Creates and analyzes individual or group crying practices monitored by smartphone or wearable device.

4. Creates and analyzes individual or group laughter practices monitored by smartphone or wearable device.

5. Creates and analyzes walking practices monitored by a smartphone or wearable device.

The stated problem is achieved through the implementation of a computer-implemented method of conducting group online practices, containing the following steps:
the user's voice is recorded by the user's device;
sending the recording from the user's device to the server for processing or performing processing locally on the user's device in order to assign the user to at least one group for performing practices;
creating at least one practice for a group of users based on data received from the user;
carry out the execution of the created practices in the group, and the execution of the practices is monitored by at least one user device or at least one wearable device of the user from the group and data is collected when performing the practices;
Based on tracking data, data is analyzed through artificial intelligence algorithms and provides users with personalized recommendations;
In a particular embodiment of the proposed method, practices are selected from the group: audio practice or video practice or conscious movement practice;
In a particular embodiment of the proposed method, based on the data obtained, such practices as gibberish and dialogue with the user are formed;
In a particular embodiment of the proposed method, users from the group are assessed with positive or negative signs for further analysis, in order to determine the reasons for a particular reaction to the user, and suggest practices based on metadata.

### BRIEF DESCRIPTION OF DRAWINGS

The implementation of the invention will be described further in accordance with the accompanying drawings, which are presented to explain the essence of the invention and in no way limit the scope of the invention. The following drawings are attached to the application:

FIG. 1 illustrates the operating diagram of the proposed method.

### DETAILED DESCRIPTION OF THE INVENTION

The invention contains three categories of digital health technology practices: audio practice, video practice, and conscious movement practice.

Audio practice is a group meditation session in an online format. Multiple users can simultaneously connect to the program and participate in a shared audio meditation practice. These methods involve transmitting audio signals from one user to another. In this way, unity and a sense of belonging to a social group are achieved.

When entering the program, the user can record from the user's device (for example, but not limited to, a smartphone, smart watch, laptop) an audio signal that represents a greeting for introduction, this can be a universal phrase or word, or sound, or a personalized greeting that the user wants to say when connecting This audio signal from the user's device is transmitted to the server, where the user's audio signal is combined into a single stream with other audio signals from other users and returned to all users simultaneously. In another embodiment of the proposed solution, the audio signal is processed locally on the user's device.

When entering the program and after recording a welcome audio signal, joining users are divided into groups where they hear a limited number of voices. For example, if 500 users are connected, they are divided into groups of 40 users. The division can be done randomly or based on certain parameters, such as, but not limited to: voice tone, gender, age, how long the person has been participating in the practice, etc. Then, from the entire group, a few voices are louder than the rest (for example, 10 out of 40 users). The optimal number of voices for groups is determined. If there are not enough voices, then at least one user's voice can be used in other groups, but the user himself is present in only one group. The choice of voices can be carried out according to the following parameters, but not limited to: combination of voice timbre, user experience.

In selected groups, the sound of voices can be carried out according to two principles:
1) The “walking microphone” principle. There is a rotation of voices that sound louder than others within the group;
2) The principle of a “static microphone”. Voices that sound louder do not change.

Users can also view the identities of other members.

A sense of shared presence is a key feature of this solution. The types of audio practices offered by the program include the practices of Special Singing and Gibberish-all of these actions are performed in a group and have a beneficial effect on the psychological state of users.

Video practice-involves interaction between users in the form of matching partners based on data called “Emotional Roulette” (Emo-Roulette), which can be carried out individually or in groups of up to 20 people. Before the session, the user must record a short video message. In the proposed solution, users, as in audio practice, are divided into groups and groups are added to specific rooms. The breakdown occurs randomly or according to a set of parameters.

The system uses user parameters to assess emotional states and associate them with those users who can best help identify suppressed emotions or evoke positive emotions. During a session, users can switch places to achieve the highest level of emotional connection.

The user parameters for assessing the emotional state are the emotions read from the user (physiognomy). Reading emotions can occur through sensors (pulse, electrical impulses of the body and other physiological indicators) or based on video and audio data (facial expressions, gestures, eye movements, voice, speech, etc.). The assessment of the emotional state consists of the fact that during and after the practice the user leaves his assessment of the practice: liked it/didn't like it/request a consultation with the leader of the practice/describe the feelings from the practice. The user can also leave his reaction to other participants: was it comfortable with them in the group/was someone annoying/someone had a positive influence, etc. The user himself describes his sensations, emotions and state during and after practice.

If there is accumulated information about the user about his emotional state, then the current parameters are compared with previously accumulated data to determine his emotional state and the dynamics of its changes during the meditation process.

Users are associated by emotional state, for example, but not limited to, users with severe aggression are associated with users with severe aggression or, for example, with a user who displays the emotion of joy.

The system continually improves its ability to accurately group users by learning and combining data about users' emotions. At the end of the session, users can express gratitude to their interaction partners and possibly initiate further communication through messaging if there is a match.

The types of video practices offered by the program include gibberish, crying, and laughing practices.

Conscious movement practice is an active practice that involves walking both indoors and outdoors. The key and distinctive aspect of this practice is the requirement to repeatedly pronounce certain sounds while walking, such as “one-two”, “poo-to”, “sat-nam”, etc. Conscious walking practices that involve the synchronous repetition of certain sounds and movement can have beneficial effects on the brain and overall well-being. For example, synchronizing walking with the monotonous speech of repeated words can stimulate the amygdala, an area of the brain involved in processing and regulating emotions. Additionally, synchronizing the right and left hemispheres of the brain through these practices can improve communication and coordination between the two hemispheres, potentially leading to improved cognitive function and mental balance.

Other practices can also be considered movement practices, such as Kundalini meditation or Sufi chakra breathing, among others.

The key difference and application of the technology is that the practice must be done using a headset so that the user can hear other users as well as the music. Using the headset, the entire biological trace is recorded, which can be collected through the microphone and the user's device (gyroscope, etc.). Voice and breathing are analyzed and compared before and after practice, as well as over time. Users do not see each other, but perform the practice simultaneously, which creates group dynamics and a sense of belonging to the group. Moreover, the user can hear the sounds made by other users during practice, thereby maintaining group dynamics.

Movement practices include any active practice that can be done together and with a headset.

All practices involve collecting data from users in different contexts, analyzing and interpreting it in relation to past data, and creating personalized recommendations for users.

For example, but not limited to, data from users are:
Inhale-through the nose/through the mouth;
Exhale-simple quiet exhalation, with a sniffle as if breathing in, loud with sound;
Sighs-need to study this, it seems this is a lack of napping;
Pitch of voice-high-tension, low-deep relaxation;
Yawning-a parameter indicating relaxation;
Pauses-sometimes there are moments of silence when a person forgets what he is doing and contemplates or falls asleep;
Length of singing-watch for changes at the beginning, at the end;
Salivation-profuse or not;
Swallowing saliva-frequency;
Inhalation/exhalation speed-length/speed;
Distractions-moving, scratching, rustling, touching, (if audio is caught);
Belching and gas-often a strong relaxation may occur, facilitating the release of gases (if we can recognize the audio);
Hoarse voice/bright tone of voice;
Singing from the stomach or from the lungs;
Accelerometer and head swing. Spacial audio.

The obtained data is analyzed with states obtained in the past through comparison using mathematical models through qualitative and quantitative changes in the user's state.

Qualitative data includes, but is not limited to: the presence or absence of yawns, wheezing, pauses in singing, movements and their nature, swallowing saliva, shortness of breath.

Quantitative data include, but are not limited to: length of inhalation and exhalation, breathing rhythm; voice volume; length and rhythm of steps; frequency of breath sampling when singing, etc.

As a result of this analysis, information about changes in the user's state is obtained. For example, that during audio practice his breathing became more rhythmic, his breaths became deeper, his voice stopped trembling, etc. In practice, this happens by comparing the time intervals of entry and exhalation, voice strength in decibels, and assessing the spectrogram of the voice. Based on video practice, the video sequence or data from the accelerometer and pedometer in the smartphone are evaluated to determine the parameters of changes in the user's state, such as, but not limited to: the fact of shaking the head and its rhythm, blinking frequency; duration of the user's stay with open and closed eyes; body swings and their rhythm; shaking, scratching; as well as other involuntary movements and their frequency of occurrence.

During movement practice, video footage or data from the accelerometer and pedometer in the smartphone are evaluated to determine parameters for changing the user's state, such as, but not limited to: the length and rhythm of steps; frequency and rhythm of breathing; duration of inhalation and exhalation; pronouncing any sounds, etc.

To create personalized recommendations, the analysis results are compared with the results of other users. They determine who made the most progress with similar starting parameters. They provide (impersonal) references to these people and offer patterns of their practices.

Also, the proposed solution involves collecting and analyzing data on the user's personal health parameters from wearable devices and other third-party solutions. For example, but not limited to, this could be:

1. Data that can be obtained from wearable devices: heart rate, sleep pattern, activity (workouts, steps taken), etc.

2. Data from connected devices: for example, electroencephalogram of the brain, etc.

3. Data provided by the user: weight, height, etc.

The received data is processed on the server or locally on the user's device. Processing includes tracking changes in parameters, including to assess the effectiveness of practices and create recommendations for practices. That is, based on this data, progress (changes) for a specific user is monitored, as well as its comparison with similar data from other users.

With a wide range of methods and the ability to track and analyze users' health data, SELF is a powerful tool for improving people's physical and emotional well-being and gaining knowledge about the personal body and mind.

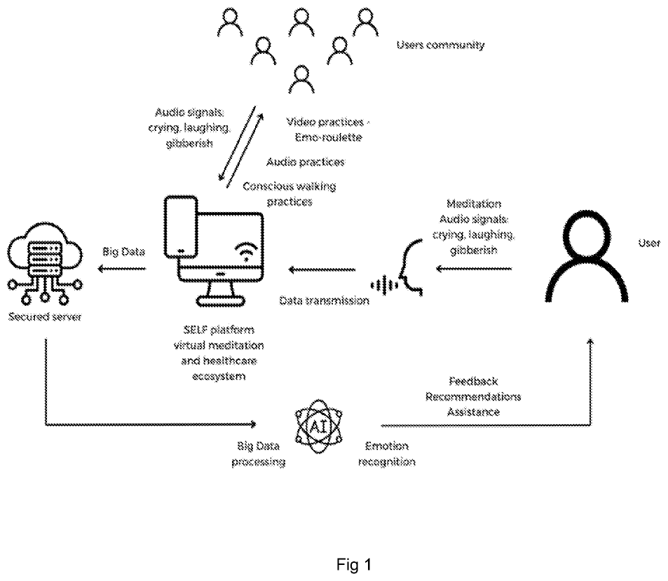

The proposed solution is carried out as follows, according to FIG. 1.

The user, through the user's device, accesses the SELF platform, which is an ecosystem of virtual meditation and health support, in the graphical interface of the user's device.

The user records a greeting audio signal and this audio signal is sent to the server or processed locally on the user's device.

Data is processed in real time in order to group people into practice, also in order to analyze the progress of individual users, compare with others, and make recommendations. Data from external devices, as well as Audio signals (crying, laughing, Gibberish, breathing) are transmitted to the platform and collected in a secure big data cloud storage. Big data is processed by artificial intelligence, thanks to which the system analyzes the user's emotional state and can provide feedback, personal recommendations related to health and psychological peace, and provide assistance and support to the user. Personal support and group practices significantly improve the psychological and physiological health of the user.

The received data is sent to a platform. Many users, who are subsequently united into groups and carry out group practices so that users have a sense of the physical presence of the group, connect to the platform.

Users are divided into groups based on the processed audio signal data. User data is collected to create personalized practice recommendations. Various practices created for a group of users (audio practice, video practice (with the ability to randomly select a partner for group practice through emotional roulette), conscious movement practice). Such practices as gibberish and dialogue with the user, singing practice and others are created.

The proposed solution also creates user avatars to represent the user in the virtual meditation space. Avatars can be used to indicate the user's presence in an audio or movement practice as desired by the user.

The avatar is created by the system in the mode of recommendations to the user. Recommendations are made based on input data about the user, as well as data collected during the internship. Avatars can be animalistic or humanoid. Avatars can reflect the characteristics of a person's behavior in practices and the emotions he expresses (calmness, aggression, joy, etc.), as well as features of his appearance.

Gibberish is randomly generated. Dialogue with the user implies the user's gibberish response to the gibberish received from the system. This practice has shown the effectiveness of improving cognitive abilities and reducing stress levels in people.

The singing practice is selected by the system from available list, for example, but not limited to “OM”. The choice of singing practice depends on the analysis of the user's tracked data (comparing current data with historical data).

The proposed solution tracks the history of practices and achievements. It is possible to share achievements with other users. Share achievements and news about the application on social networks (Facebook, Instagram, Twitter) and instant messengers (Telegram, WhatsApp, etc.).

The proposed solution includes the ability to invite new users to the system and invitations to created user groups.

he proposed solution also evaluates users with positive or negative signs for further analysis, in order to determine the reasons for a particular reaction to the user, and propose practices based on metadata. This assessment is carried out by system users during the practice. This assessment can be carried out on the basis of whether the user likes/dislikes or with the ability to mark one's emotions towards the user (for example, but not limited to, caused anger, irritation, joy, admiration, etc.).

A computing system that provides data processing necessary to implement the claimed solution generally contains components such as: one or more processors, at least one memory, data storage, input/output interfaces, input means, network communication means. When executing machine-readable instructions contained in the RAM, the device processor is configured to perform basic computing operations necessary for the operation of the device or the functionality of one or more of its components. The memory is usually in the form of RAM, into which the necessary program logic is loaded to provide the required functionality. When implementing the proposed solution, the necessary amount of memory is allocated to implement the proposed solution is allocated. Data storage media can be presented in the form of HDDs, SSD drives, raid arrays, network storage, flash memory, etc. The tool allows long-term storage of various types of information. Interfaces are presented as standard means for connecting and operating peripheral and other devices, for example, USB, RS232, RJ45, COM, HDMI, PS/2, Lightning, etc. A keyboard, joystick, display (touch display), projector, touchpad, mouse, trackball, light pen, speakers, microphone, etc. can be used as data input means. The means of network interaction are selected from a device that provides network reception and transmission of data, for example, an Ethernet card, WLAN/Wi-Fi module, Bluetooth module, BLE module, NFC module, IrDa, RFID module, GSM modem, etc. The tools ensure the organization of data exchange over a wired or wireless data transmission channel, for example, WAN, PAN, LAN, Intranet, Internet, WLAN, WMAN or GSM. The device components are interconnected via a common data bus.

In these application's materials, a preferred disclosure of the implementation of the claimed technical solution was presented, which should not be used as limiting other, private embodiments of its implementation, which do not go beyond the scope of the requested scope of legal protection and are obvious to specialists in the relevant field of technology.

What is claimed is:

1. A computer-implemented method of conducting group online practices, containing stages in which:
the user's voice is recorded by the user's device;
the recorded voice of the user from the user's device is sent to the server for processing or performing processing locally on the user's device in order to assign the user to at least one group for performing practices;
at least one practice for a group of users based on the data received from the user is created;
the execution of the created practices in the group is carried out, and the execution of the practices is monitored by at least one user device or at least one wearable device of the user from the group and data is collected when performing the practices;
based on tracking data, data is analyzed using artificial intelligence algorithms and provide users with personalized recommendations.

2. The method of claim 1, differing in that practices are selected from a group: audio practice or video practice or conscious movement practice.

3. The method of claim 1, differing in that based on the data obtained, practices such as gibberish and dialogue with the user or the practice of singing are formed.

4. The method of claim 1, differing by the fact that users from the group are assessed with positive or negative signs for further analysis, in order to determine the reasons for a particular reaction to the user, and to propose practices based on metadata.
